(19) United States
(12) Patent Application Publication
(10) Pub. No.: US 2003/0097167 A1
(43) Pub. Date: May 22, 2003

(54) Transesophageal cardiac probe and methods of use

(76) Inventor: Paul A. Friedman, Rochester, MN (US)

Correspondence Address: Richard J. Anderson, Fish & Richardson P.C., P.A., Suite 3300, 60 South Sixth Street, Minneapolis, MN, 55402, US

(21) Appl. No.: 10/293,685

(22) Filed: Nov. 13, 2002

Related U.S. Application Data
(60) Provisional application No. 60/338,232, filed on Nov. 13, 2001.

(51) Int. Cl. A61N 1/05

(52) U.S. Cl. 607/124

(57) ABSTRACT

The invention provides for an esophageal probe for transesophageal cardiac stimulation. An esophageal probe can be made de novo or can be a modified transesophageal echocardiogram (TEE) probe. The invention further provides for an electrode-containing membrane to so modify a TEE probe for transesophageal cardiac stimulation. Methods are provided by the invention for using esophageal probes of the invention for transesophageal pacing or defibrillation.

### CROSS REFERENCE TO RELATED APPLICATIONS

This application claims priority under 35 U.S.C. §119(e) of U.S. Application No. 60/338,232, filed Nov. 13, 2001.

### TECHNICAL FIELD

This invention relates to cardiac arryhthmias, and more particularly to a transesophageal probe for cardiac stimulation and methods of using such a probe.

### BACKGROUND

Atrial fibrillation is the most common sustained arrhythmia, affecting more the 2 million Americans. It is also the most common rhythm disturbance necessitating hospital admission at an estimated annual cost of $1 billion dollars. Arrhythmias are associated with an increased risk of stroke, and heart failure. The annual stroke rate for patients with atrial fibrillation is increased 4-6 fold compared to the normal population, with thromboembolism accounting for the majority of cerebrovascular events. Management of atrial fibrillation involves prevention of thromboembolism with anticoagulants and symptomatic treatment of the arrhythmia itself, with cardioversion used to terminate episodes. Prior to cardioversion, transesophageal echocardiography is often performed to exclude the presence of intra-atrial thrombus, and to assess cardiac function and anatomy. Transesophageal echocardiography (TEE) is a common clinical procedure in which a transducer-tipped probe is passed down the esophagus in close proximity to the heart for enhanced ultrasonic imaging.

The clinical impact of atrial fibrillation is large. In 1998, patients underwent an estimated >100,000 cardioversions to treat arrhythmia. Approximately half of all patients underwent TEE immediately preceeding or during cardioversion. Clearly, a technique that can terminate atrial tachyarrhythmias without the pain of a shock (and its attendant need for general anesthesia), or which can increase the likelihood of successful cardioversion would greatly enhance patient care.

There has been interest in the development of painless methods to promptly terminate atrial fibrillation. Antitachycardia pacing (ATP) has been highly effective in isthmus-dependent (typical) atrial flutter, which is a highly organized rhythm with a large excitable gap. ATP has not been effective, however, in treating disorganized rhythms such as atypical flutter or atrial fibrillation. It has been demonstrated that atrial fibrillation has an excitable gap. This means that there is excitable atrial tissue between wandering fibrillatory wavefronts. If a pacing impulse could excite this tissue before the next fibrillatory wavefront enters it, then the wavefront could be extinguished for want of excitable tissue to propagate onto. Indeed, it has been shown that high frequency burst pacing can capture up to a 4 cm region of the atrium in a fibrillating animal model.

### SUMMARY

The invention provides for an esophageal probe for transesophageal cardiac stimulation. An esophageal probe can be made de novo or can be a modified transesophageal echocardiogram (TEE) probe. The invention further provides for an electrode-containing membrane to so modify a TEE probe for transesophageal cardiac stimulation. Methods are provided by the invention for using esophageal probes of the invention for transesophageal pacing or defibrillation.

In one aspect, the invention provides for an esophageal probe for transesophageal cardiac stimulation, including: (a) an elongated flexible member having a distal portion and a proximal portion, wherein the distal end is closed for inserting into the esophagus; and (b) a plurality of conductive electrodes, wherein the plurality of electrodes are circumferentially disposed at least partially around the distal portion of the elongated member.

A probe of the invention can further include at least one conductor extending from the proximal portion to the plurality of electrodes. A probe of the invention can further include a circuit, a pulse generator, and/or a selector means. The selector means can be used to select and energize particular electrodes from the plurality of electrodes. A probe that includes a pulse generator can further include a control unit. A control unit can selectively vary at least one characteristic of a pulse from the pulse generator such as the waveform, the duration of the pulse, the interval between pulses, and the sequence of pulses to various electrodes.

The plurality of electrodes can be connected to one another or can be distinct from one another. One or more of the plurality of electrodes can function as pacing electrodes, defibrillation electrodes and/or sensing electrodes. Pacing electrodes typically conduct electrical current at about 1 milliamp (mA) to about 20 mA, while defibrillation electrodes generally emit energy of from about 1 Joule (J) to about 100 J. Generally, each of the plurality of electrodes are spaced longitudinally apart at a distance of from about 5 mm to about 5 cm. A probe of the invention can further include a temperature sensor at the distal end and at least one conductor connected to the temperature sensor.

In another aspect of the invention, there is provided an improved transesophageal echocardiogram probe including: a housing having a cylindrical cavity formed therein; an elongated, multi-element ultrasonic array, the array including a number of elongated piezoelectric elements having emitting surfaces arranged in a plane, the array having a scan axis which is perpendicular to the long axis of the elements, the array being mounted on a pulley within the cavity; means for rotating the pulley within the housing, whereby the array will be rotated relative to the housing and in the plane of the elements around the axis of rotation of the pulley; means adapted for connecting the means for rotating to an operator control remote from the housing; and means for electrically connecting the array to an external ultrasound unit, wherein the improvement includes: a plurality of conductive electrodes, wherein the plurality of electrodes are circumferentially disposed at least partially around a distal portion of the housing.

An improved transesophageal echocardiogram probe of the invention can further include at least one conductor extending from a proximal portion of the housing to the plurality of electrodes. Typically, the plurality of electrodes can be connected to one another or can be distinct from one another. Further, the plurality of electrodes can be disposed on a disposable membrane.

It is another aspect of the invention to provide a pacing or defibrillating member usable with a transesophageal probe, the member including: a flexible, planar sheet member including a plurality of spaced apart electrical conductors; a layer of adhesive carried on the sheet member; and a removable protective cover overlaying the adhesive layer, wherein the flexible, planar sheet member is configured for overlaying onto the transesophageal probe. Such a flexible planar sheet member can further include a plurality of spaced apart planar electrodes, wherein the plurality of spaced apart planar electrodes corresponds positionally to the plurality of spaced apart electrical conductors. A pacing or defibrillating member of the invention can further include a plurality of planar electrodes and/or low impedance defibrillating electrodes. The electrodes can be pacing electrodes. According to the invention, the member can be disposable.

In yet another aspect of the invention, there is provided a method for treating atrial fibrillation, including the steps of: a) inserting a flexible probe into the esophagus of an individual at a position within the esophagus wherein the probe is adjacent to the atrium of the individual's heart, wherein the probe includes a plurality of electrodes connected to a pacing generator via at least one conductor; and b) generating pacing pulses in the pacing generator, c) transmitting the pacing pulses from the generator to one or more of the electrodes via the at least one conductor; and d) transmitting the pacing pulses from the electrode(s) through the wall of the esophagus to the individual's atrium, thereby treating the atrial fibrillation in the individual.

Pacing pulses administered to treat atrial fibrillation can be administered in a range of from about 75 mA to about 150 mA. In addition, pacing pulses can be administered at a rate of from about 70 to about 100 pulses per minute. Further, pacing pulses can be administered at a frequency of 50 Hz.

In another aspect of the invention, there is provided a method of terminating atrial fibrillation in an individual's heart, including the steps of: a) positioning a plurality of conductive electrodes in the individual's esophagus adjacent to a posterior surface of the heart; and b) pulsing selected of the conductive electrodes with electrical signals of a predetermined frequency thereby terminating the atrial fibrillation

In yet another aspect of the invention, there is provided a method of cardioversion, including the steps of: a) inserting a TE probe into the esophagus of an individual in need of cardioversion; and b) emitting an electrical signal from the TE probe, wherein the electrical signal results in cardioversion. In addition, a cardiac image on the individual prior to or concurrent with the emission of the electrical signals can be obtained. Generally, a signal is ascending in voltage, is biphasic, and is a rounded biphasic ascending ramp.

In another aspect, the invention provides a system for performing electrophysiological testing on an individual, including: a) a transesophageal probe of the invention; b) a pulse generator and receiver means connected to at least two of the plurality of electrodes for delivering pulses to selected electrodes and for receiving electrical signals induced in selected electrodes; c) control means connected to the pulse generator and receiver means for controlling generation of the pacing pulses; d) monitoring means connected to the pulse generator and receiver means for displaying data representative of parameters of electrical signals induced in at least one of the plurality of electrodes. The system can further include means for displaying an electrocardiogram representing sensed electrical activity of the heart.

In another aspect, the invention provides a computer readable storage medium having instructions stored thereon causing a programmable processor to: (1) determine local bipolar cycle lengths, minimum cycle length, maximum cycle length and mean cycle length over 5 sec; (2) administer electrical signal beginning at maximum cycle length and, over 2 sec, accelerate to the mean cycle length which is maintained for 2 sec; (3) repeat step (1); (4) if atrial fibrillation is present, continue to step (5); if atrial fibrilliation is absent, stop; (5) administer an electrical signal beginning at maximum cycle length and, over 2 sec, accelerate to a cycle length ½ the distance from the mean to the minimum cycle length which is maintained for 2 sec; (6) repeat step (1); (7) if atrial fibrillation is present, continue to step (8); if atrial fibrillation is absent, stop; (8) administer an electrical signal beginning at maximum cycle length and, over 2 sec, accelerate to the minimum cycle length which is maintained for 2 sec; (9) repeat step (1); and (10) if atrial fibrillation is present, repeat steps (8), (9) and (10); if atrial fibrillation is absent, stop.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DESCRIPTION OF DRAWINGS

FIG. 1 shows an image of an embodiment of a transesophageal probe of the invention.

FIGS. 2A-C show images of an embodiment of a transesophageal probe of the invention. FIG. 2D is a schematic showing construction of an electrode-containing member for use with a transesophageal echocardiogram probe.

FIG. 3 is a schematic of the electrodes that can be used on a transesophageal probe of the invention.

FIG. 4 is a schematic of the electrical connections configured for recording/pacing or shocking (cardioversion).

FIG. 5 is a schematic of a band electrode that can be used on a transesophageal probe of the invention.

FIG. 6 shows simulations of the pacing algorithm using randomly generated cycle lengths as input.

Like reference symbols in the various drawings indicate like elements.

### DETAILED DESCRIPTION

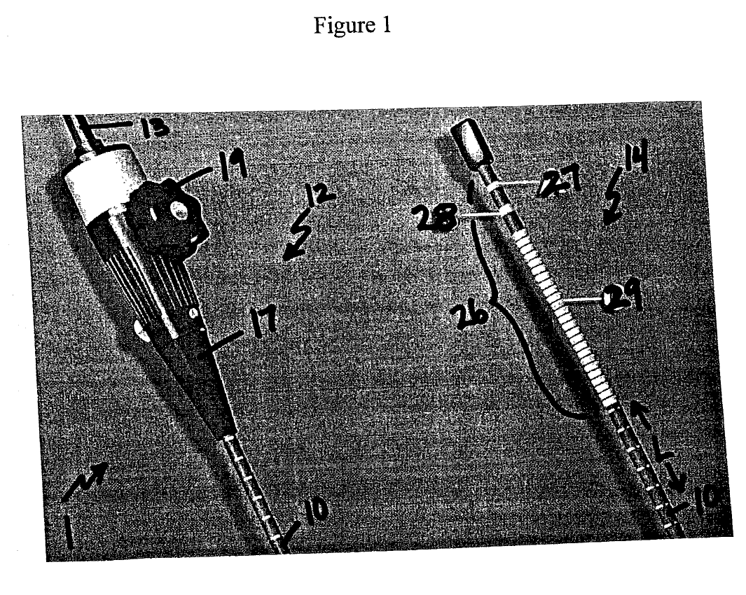

Referring to FIG. 1, an embodiment of a transesophageal probe 1 is shown that includes an elongated flexible member 10. The elongated flexible member 10 includes a proximal portion 12 and a distal portion 14 along a longitudinal axis L of the probe 1. The distal portion 14 of an elongated flexible member 10 is generally closed for insertion into the esophagus. The elongated flexible member can be tubular for carrying a conductor (not shown) from an array of electrodes 26 mounted on the distal portion 14 of the elongated flexible member 10 and attached to a connector 13 at the proximal portion 12 of the elongated flexible member 10. Alternatively, a conductor can be external to the elongated flexible member 10.

The proximal portion 12 of an elongated flexible member 10 typically contains a handle 17 to be grasped by a user. Configurations and elements required of a probe handle are well known in the art. A proximal portion 12 of an elongated flexible member 10 can further contain a mechanism 19 to be manipulated by the user to control the distal portion 14 of the elongated flexible member 10. In addition, the proximal end of an elongated flexible member can be connected via a connector 13 to, for example, a pulse generator, a circuit, or a control unit (not shown). A pulse generator, a circuit, or a control unit can act as an energy source or can regulate or control the energy delivered to the electrodes.

The proximal 12 and distal 14 portions of the elongated flexible member 10 can be integrally formed from a biocompatible material having requisite strength and flexibility for introducing and advancing the transesophageal probe 1 of the invention into the esophagus of an individual. The proximal 12 and distal 14 portions can be flexible to facilitate articulation of a transesophageal probe 1 during use. Appropriate materials are well known in the art and generally include polyamides such as, for example a woven material available from DuPont under the trade name Dacron.

Annular electrodes 26 are circumferentially disposed about the distal portion 14 of the elongated flexible member 10. Electrodes can be for pacing procedures or for cardioversion procedures. FIG. 1 shows an elongated flexible member 10 having a distal electrode for bipolar recording and pacing 27, a proximal electrode for bipolar recording and pacing 28, and a large surface area electrode 29 for transesophageal defibrillation.

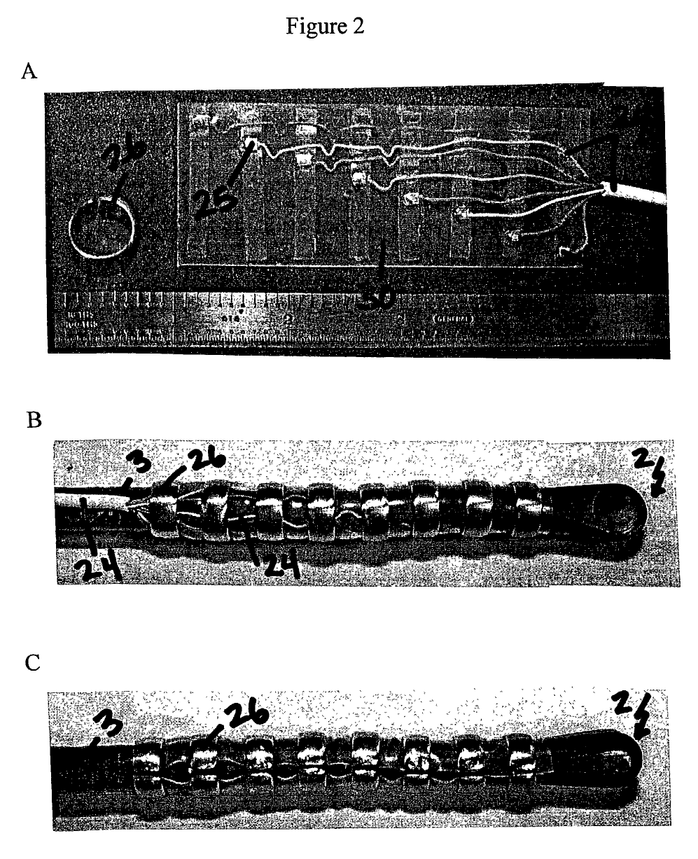

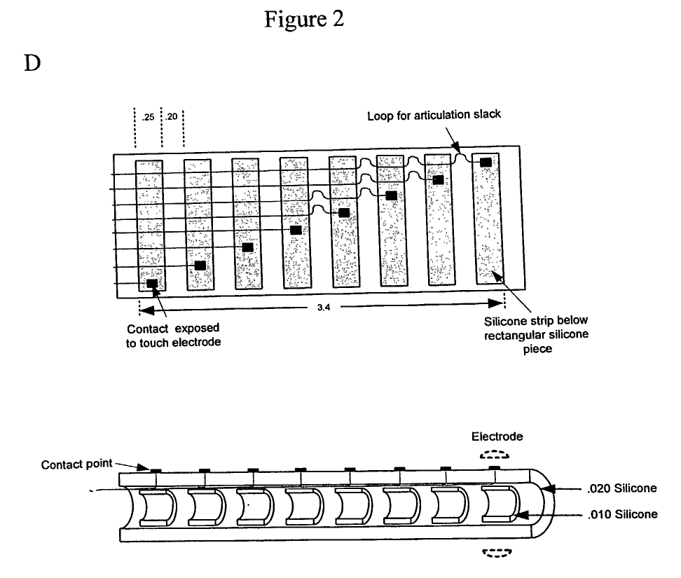

Referring to FIG. 2A, the electrode rings 26 and a silicone sheet subassembly 30 containing electrical contacts 25 and conductors (e.g., wires) 24 to each contact are shown. FIGS. 2B and 2C show a finished electrode assembly attached to a probe. Eight stainless steel (300 series) electrodes 26 can be clamped onto an ultrasound or TEE probe 3 over a silicone sheet subassembly 30 to generate a transesophageal echocardiogram probe that can be used for cardiac stimulation 2.

FIG. 2D is a schematic that shows the details of a silicone sheet subassembly 30. Wire is passed through the top layer of silicone with a contact point exposed. A silicone strip is placed over each wire. The silicone strips are secured with adhesive silicone and can act as a fixing mechanism for the wires as well as an aiding the connection between the contact point and the electrode. An ‘articulation loop’ for the first four wires is placed between the electrodes. This assembly is wrapped around the distal portion 14 of a TEE probe and the electrodes are crimped on in positions that correspond to the electrical contacts. Wire conductors 24 can be made from 28-gauge copper, with 0.003 inches of teflon insulation. Breakdown voltage for the wire is 1800 volts.

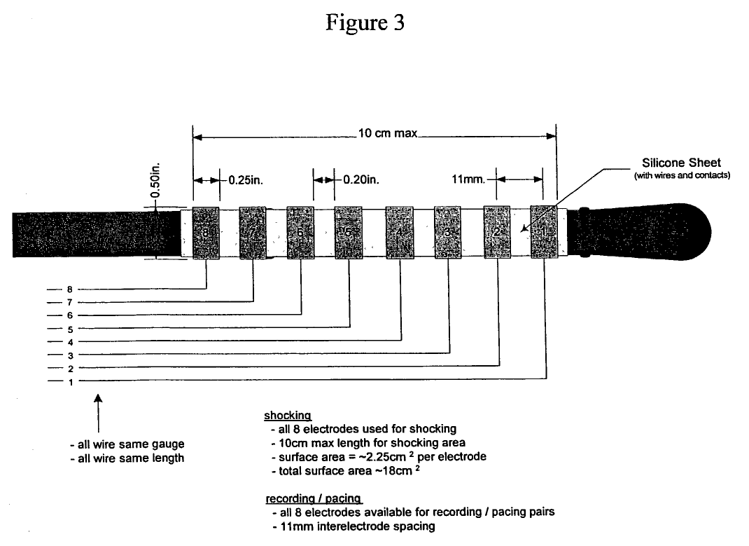

As shown schematically in FIG. 3, electrodes for permanent affixation to a transesophageal probe or for attachment via a pacing/defibrillation member can be about 0.25 inches wide, 0.5 inches in diameter, with a 0.20-inch spacing between electrodes.

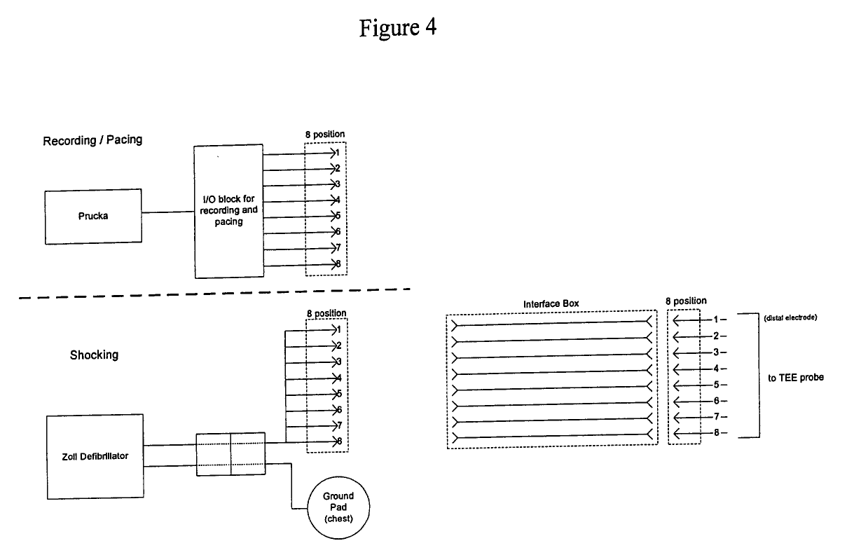

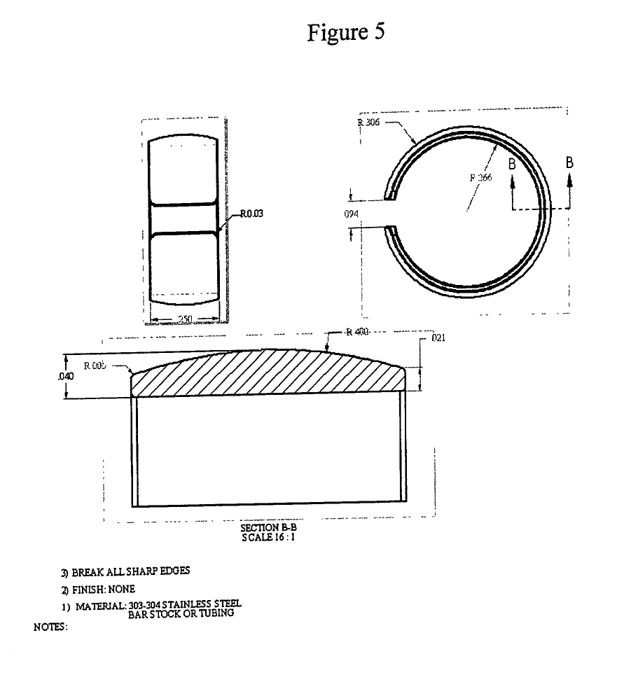

FIG. 4 schematically shows connections that permit electrode pairs to be selected for pacing and/or recording and further permit all eight electrodes to be used together for cardioversion. FIGS. 2-4 show transesophageal probes or silicon sheet subassemblies having eight electrodes. More or less electrodes can be used on a transesophageal probe for transesophageal cardiac stimulation. The number of electrodes used for transesophageal cardiac stimulation will depend upon their size, the type of energy they emit, and the energy source for the electrodes. FIG. 5 shows a schematic of a single electrode.

Pacing of the left atrium can be performed from within the esophagus due to the juxtaposition of these two structures. Transesophageal therapy may have several benefits, including the possibility of arrhythmia termination without general anesthesia. In the past, the transesophageal echo probe has been used exclusively as a diagnostic tool. However, the probe's position within the esophagus in close proximity to the heart permits the delivery of new therapeutic interventions. The addition of electrodes to the probe to permit delivery of pacing and cardioversion therapies.

Low energy high frequency pacing can be used to painlessly terminate atrial fibrillation. Pacing of the left atrium can be performed from within the esophagus due to the juxtaposition of these two structures. Additionally, a rate-adaptive pacing algorithm is disclosed herein and can be employed.

Cardioversion from within the esophagus can be performed using a large surface area electrode. A biphasic waveform, designed to increase effectiveness and limit pain was developed. This biphasic waveform may permit shock delivery without general anesthesia.

Since most patients eligible for the present study will already be in atrial fibrillation, they will not be at risk for atrial rhythm deterioration. Patients with atrial flutter may experience atrial flutter degeneration to atrial fibrillation that then fails to respond to pacing. In that case, patients will undergo cardioversion.

Clinical trials of high frequency burst pacing in atrial fibrillation have been disappointing. All clinical studies to date, however, have evaluated 50 Hertz burst pacing rates. Using this approach, a standard rapid pacing rate of 50 Hz is applied, irrespective of the arrhythmia rate. The failure of the pacing rate to match the arrhythmia rate may seriously impair the pacing impulses' ability to penetrate the excitable gap and terminate the arrhythmia. However, use of a rate adaptive algorithm, in which each pacing burst rate is specifically tailored to the individual arrhythmia episode, may improve pacing success rates. By matching the pacing rate to the atrial fibrillation rate (by means of a statistical analysis of the fibrillation), a pacing algorithm may more effectively penetrate the excitable gap and terminate the arrhythmia.

Review of intracardiac electrograms and published animal data indicates that the pacing rate is critical for successful regional capture of myocardium during atrial fibrillation. Pacing at rates that are too slow permit a wandering atrial fibrillation wavefront to spread over the pacing region between pacing impulses, preventing local capture by a pacing electrode. Conversely, pacing at a rate that is too fast may result in local reinitiation of atrial fibrillation. It is critical, therefore, that the pacing rate be well matched to the atrial fibrillation to prevent local loss of capture and to prevent re-initiation of atrial fibrillation. Thus, a new algorithm, designed to permit delivery of a rate adaptive burst during atrial fibrillation unique to the individual episode has been developed.

The following steps describe the pacing algorithm of the invention.

Step 1—After atrial fibrillation is present, during a 5 second period, local bipolar cycle lengths are measured, and the minimum, maximum, and mean cycle length determined.

Step 2—The initial high frequency burst pacing begins at the maximum cycle length and over a period of 2 second accelerates to the average cycle length which is maintained for 2 seconds.

Step 3—Local cycle lengths are measured over a 5 second interval, local bipolar cycle lengths are measured, and the minimum, maximum, and mean cycle length are re-determined. If atrial fibrillation is terminated, the algorithm is complete. If not, it proceeds to Step 4.

Step 4—High frequency burst pacing again, beginning at the maximum cycle length and over a period of 2 second accelerates to a cycle length ½ the distance from the average to the minimum AF cycle length, and maintain this pacing rate for a period of 2 seconds.

Step 5—Local cycle lengths are measured over a 5 second interval, local bipolar cycle lengths are measured, and the minimum, maximum, and mean cycle length are re-determined. If atrial fibrillation is terminated, the algorithm is complete. If not, it proceeds to Step 6.

Step 6—High frequency burst pacing again, beginning at the maximum cycle length and over a period of 2 second accelerates to the minimum AF cycle length, and maintain this pacing rate for a period of 2 seconds.

Step 7—Local cycle lengths are measured over a 5 second interval, local bipolar cycle lengths are measured, and the minimum, maximum, and mean cycle length are re-determined. If atrial fibrillation is terminated, the algorithm is complete. If not, maximum pacing rate is to AF minimum cycle length.

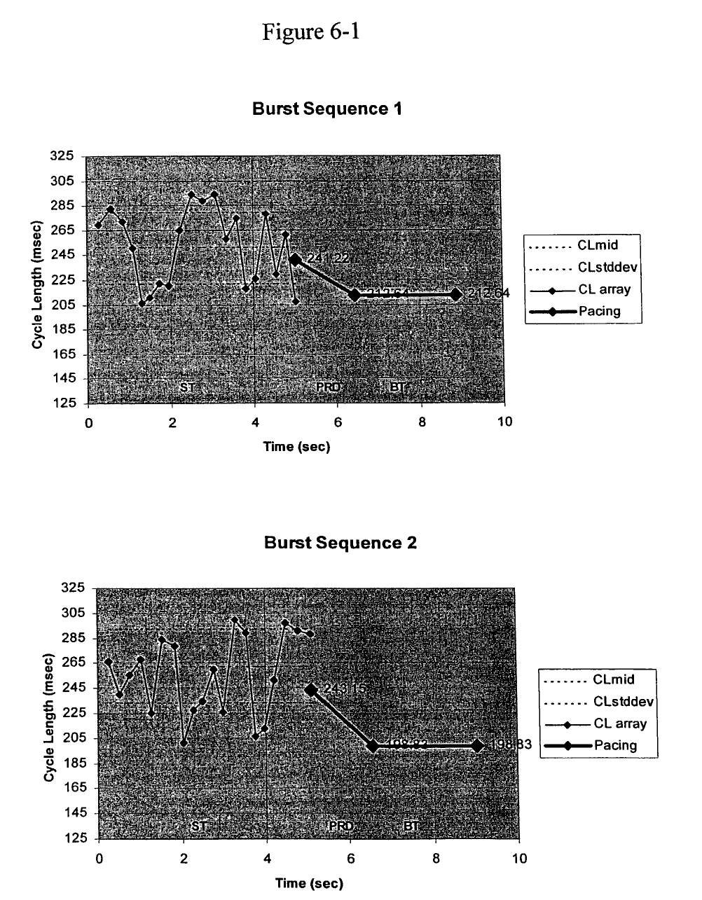

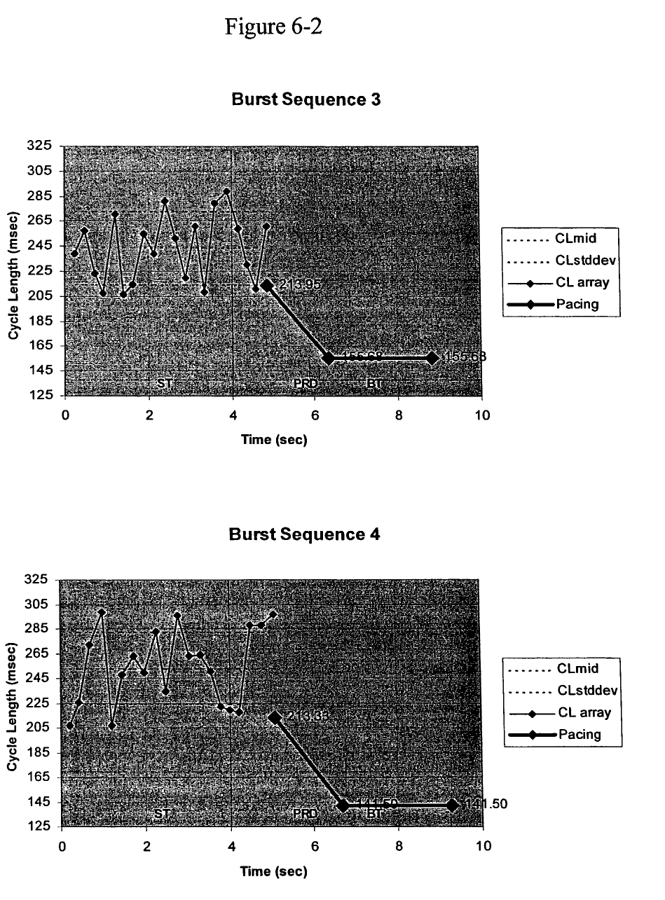

The graphs shown in FIG. 6 illustrate a simulation using the pacing algorithm disclosed herein. Input to the algorithm is a set of randomly generated cycle lengths between 200 and 300 msec.

The invention also provides for a system to carry out transesophageal cardiac stimulation. Such a system includes a transesophageal probe as disclosed herein, a pulse generator and receiver means connected to at least two of the plurality of electrodes, control means connected to the pulse generator and receiver means, and monitoring means connected to said pulse generator and receiver means. The pulse generator delivers pulses to selected electrodes and receives electrical signals induced in selected electrodes. The control means controls the pacing pulses, and the monitoring means displays data that is representative of the parameters of the electrical signals that are induced in at least one of the plurality of electrodes.

The invention will be further described in the following examples, which do not limit the scope of the invention described in the claims.

### EXAMPLES

### Example 1

Experimental Protocol for Evaluating Pacing Via a TE Probe

Patients referred to the Cardioversion Center at the Mayo Clinic (Rochester, Minn.) for a clinically indicated TEE and cardioversion are eligible to participate. Patients are prospectively randomized into one of two arms:

a) TEE pacing: if the patient is still in atrial fibrillation after 5 minutes, 1 mg of ibutilide is administered IV, followed by repeat pacing in 10 minutes if the patient is still in atrial fibrillation; for patients experiencing persistent atrial fibrillation, standard transthoracic shock is administered; and

b) Placebo infusion IV: if the patient is still in atrial fibrillation after 5 minutes, 1 mg of ibutilide is administered IV; if the patient is still in atrial fibrillation after 10 minutes, standard transthoracic shock is administered.

Patients on antiarrhythmic drugs are eligible for this protocol. This is consistent with current clinical practice and Cardioversion Center guidelines, in which antirarrhythmic drug recipients remain eligible for ibutilide. Exclusion criteria for ibutilide (as per standard Cardioversion Center protocol) include EF <30 % (EF will always be known as all patients receive TEE), QTc>480, and/or pregnancy. Clinical, structural, and hemodynamic variables, acute outcome, complications, and 3-month outcomes are collected as per standard Cardioversion Center practice.

The study arm and control arm are identical except for the absence of TEE pacing in the placebo arm. Interpretation of the results from previous studies of high frequency pacing for the termination of atrial fibrillation have been limited by the absence of a placebo control. Since atrial fibrillation can terminate spontaneously, a placebo control arm is necessary to accurately assess the impact of the pacing intervention. Ibutilide is used for two reasons: 1) administration of ibutilide is currently part of the clinical practice protocol at the Mayo Cardioversion Center; and 2) since ibutilide increases the excitable gap of atrial fibrillation (even if it does not terminate it), there is an anticipated synergy between pacing and ibutilide.

### Example 2

Traditional Pacing

In a study of patients undergoing electrophysiologic study (EPS), the efficacy of 50 Hz high frequency burst (HFB) pacing was assessed for termination of atrial flutter and atrial fibrillation. Atrial arrhythmias were induced at the time of EPS. After one minute of arrhythmia, patients received ten 30-second blocks of “therapy” (1 second of HFB followed by 29 sec of observation) or “control” (30 sec observation only) in a prospectively randomized manner using a “Latin squares” table. Termination during therapy blocks was compared to termination during control blocks, stratified on arrhythmia type. Twenty-nine episodes of atrial arrhythmia were induced in 15 patients. Atypical (non-isthmus dependent) atrial flutter as defined by surface ECG and intracardiac electrograms was induced in 9 patients (total of 18 episodes), and atrial fibrillation was induced in 6 patients. Atrial fibrillation was terminated in only one patient (one of two episodes) during HFB delivery, but persisted during all other attempts of HFB (7 atrial fibrillation episodes) or spontaneously terminated during the “control” (observation) period. In every patient with atypical flutter, at least one attempt of HFB terminated arrhythmia (15/18 episodes terminated during HFB therapy). In summary, it was found that a standard 50 Hz pacing burst was effective for treating atypical flutter, and less effective for treating atrial fibrillation.

### Example 3

Pacing Algorithm

Pretest conditions include the following: placing 3 surface ECG electrodes on a patient (typically the right shoulder, the left shoulder, and a reference position); placing a TEE probe with 2 ECG electrodes in the esophagus of a patient; and connecting the patient to an external defibrillator.

A pretest setup is typically performed. Generally, the following steps are performed. The system and associated hardware is powered on. Preliminary system diagnostics are run to verify that the system is operating correctly. The patient's name and the clinic number are entered on the User Interface. A filename for recording analog input data and logging system parameters is automatically generated. All analog data is time stamped and archived to disk for each case. All system parameters are time stamped and archived to disk and only updated if changed after that. A patient's ECG electrodes (both surface and internal) are connected to ECG amplifiers and ECG waveforms are verified on the User Interface screen. The test is aborted if the calculated midpoint atrial heart rate (HRmid) is below the detected heart rate (HRdetect). The pacer/stimulator output current is set as desired (this is performed manually at the pacer/stimulator itself, not on the User Interface).

Test pacing (i.e., manually pacing the patient with pacer/stimulator pulses and testing the electrode placement for atrial, and not ventricular, pacing) is typically performed next. Text pacing can include the following steps. Pacer/stimulator interlock is deactivated by pressing the ARM PACER button on the User Interface while holding in the hardware interlock button. The user is notified that the system is about to be armed for pacing, and will be given the option to continue or cancel.

Test pacing parameters are set on the User Interface. Test pacing rate default is 120 beats per minute (bpm) unless adjusted to another setting between 100 and 600 bpm. The test pacing duration default is 0.5 seconds unless adjusted to another setting between 0.5 and 10 seconds. The test pacing pulse width default is 15 msec unless adjusted to another setting between 5 and 25 msec. The test pacing output is activated. The test output is only active when the <Shift> key and left mouse button are both pressed and held down while the cursor is over the TEST PACER button on the User Interface. The pacer/stimulator output stops when the left mouse button is no longer pressed or the test pacing duration is reached. The test output cannot be reactivated for a minimum of 5 seconds after a test pacing output sequence has completed.

Burst pacing is then performed. Burst pacing can include the following steps. The adjustable test parameters are set on the User Interface according to Table 1. The following analog input parameters are adjusted on the User Interface until the displayed ECG measurement points for atrial fibrillation cycle times are determined to be adequate by the physician: a) input band-pass filter high frequency cutoff; b) input band-pass filter low frequency cutoff; c) input amplitude high threshold level; and d) input amplitude low threshold level.

The pacer/stimulator interlock is deactivated by pressing the ARM PACER button on the User Interface while holding in the hardware interlock button. The user is notified that the system is about to be armed for pacing, and is given the option to continue or cancel. The system is stopped at any time by pressing the HALT button on the screen using the mouse, by pressing the ESC key on the keyboard, or by pressing an external hardware Emergency Stop. This will disarm the system and return the user to the main starting screen.

A burst pacing sequence is then administered. The system calculates the midpoint atrial fibrillation cycle time (CLmid) over the selected sensing time (ST) and deliver output stimulus. The following applies: the test is aborted if the calculated midpoint atrial heart rate (HRmid) is below the detected heart rate (HRdetect); pacing bursts begin at the calculated starting cycle length (PCLstart); the cycle length decreases linearly to the calculated ending cycle length (PCLend) over the specified pacing ramp duration (PRD) time; and the ending cycle length is maintained for the specified pacing burst tail (PBT) time.

The system calculates the atrial fibrillation cycle times over the specified time interval between sequences (dTseq). If the calculated midpoint atrial fibrillation heart rate (HRmid) is still above the threshold detected heart rate (HRdetect), the user is prompted to continue to the next burst pacing sequence. The user has the option to continue or cancel. If the calculated midpoint atrial fibrillation heart rate (HRmid) is below the threshold detected heart rate (HRdetect), the system will disarm and start over at initial adjustments. The user is notified that this has occurred.

Burst pacing sequences continues until one of the following conditions is met: the calculated midpoint atrial fibrillation heart rate (HRmid) is below the threshold detected heart rate (HRdetect); the specified total number of pacing sequences (TNPS) has been executed; the user cancels the test at the prompt between sequences; or the user aborts the test with the hardware E-Stop, User Interface Halt button, or Escape key

TABLE 1
Variables and equations used in the pacing algorithm

| Code | Variable | Description | Min | Max | Default | Units |
|---|---|---|---|---|---|---|
| CL[] | Cycle Length | Array of cycle lengths measured | CLmin | CLmax | N/A | msec |
| | array | during sensing time ST | | | | |
| CLmin | Minimum | Minimum local bipolar cycle | calculated from CL[] | | | msec |
| | Cycle Length | length | | | | |
| CLmax | Maximum | Maximum local bipolar cycle | calculated from CL[] | | | msec |
| | Cycle Length | length | | | | |
| CLmean | Average | Average local bipolar cycle | calculated from CL[] | | | msec |
| | Cycle Length | length | | | | |
| CLmedian | Median Cycle | Median local bipolar cycle | calculated from CL[] | | | msec |
| | Length | length | | | | |
| CLstddev | Standard | Standard Deviation of Cycle | calculated from CLmean | | | msec |
| | Deviation of | Length array | | | | |
| | Cycle Length | | | | | |
| CLmid | Midpoint | Cycle length to use as midpoint | calculated | | | msec |
| | Cycle Length | in determining burst pacing | = CLmean or CLmedian | | | |
| HRdetect | Detection | Atrial heart rate above which | 100 | 200 | 150 | beats/ |
| | Heart Rate | pacing will be delivered. | | | | min |
| | | Heart rates below this cutoff will | | | | |
| | | be treated as normal sinus | | | | |
| | | rhythm, and no therapy will be | | | | |
| | | delivered. | | | | |
| HRmid | Midpoint | Heart rate that will be used to | calculated from CLmid | | | beats/ |
| | Heart Rate | determine whether pacing will | (if HRmid < HRdetect, then | | | min |
| | | be delivered. | no pacing) | | | |
| PCLoffset | Pacing Cycle | Cycle length offset used to | −100 | 100 | 10 | msec |
| | Length Offset | calculate Start Pacing Cycle | | | | |
| | | Length PCLstart | | | | |
| | | Number of milliseconds less | | | | |
| | | than CLmid. A negative number | | | | |
| | | will lead to starting pacing at a | | | | |
| | | heart rate with a cycle length | | | | |
| | | greater than the ongoing | | | | |
| | | arrhythmia. | | | | |
| dPCLstart | Decrement of | Decrement of Start Pacing Cycle | 0 | 50 | 10 | msec |
| | Start Pacing | Length between burst sequences | | | | |
| | Cycle Length | | | | | |
| PCLstart | Start Pacing | Starting cycle length for pacing | calculated | | | msec |
| | Cycle Length | pulses | = CLmid - PCLoffset | | | |
| | | This formula will cause pacing | [(N-1) * dPCLstart] | | | |
| | | to begin at a heart rate higher | where N = burst sequence | | | |
| | | than that of the ongoing | number | | | |
| | | arrhythmia. The rate will be | conditions: | | | |
| | | increased further during each | PCLstart ≧ PCLmin | | | |
| | | subsequent burst sequence. | | | | |
| PCLmin | Minimum | Absolute minimum Pacing | 10 | 250 | 20 | msec |
| | Pacing Cycle | Cycle Length allowed | | | | |
| | Length | | | | | |
| PCLfactor | End Pacing | Multiplication factor for | 0 | 10 | 1 | msec |
| | Cycle Length | calculation of PCLend | | | | |
| | multiplication | | | | | |
| | factor | | | | | |
| dPCLend | Decrement of | Number of standard deviations | 0 | 10 | 0.5 | N/A |
| | End Pacing | to decrement End Pacing Cycle | | | | |
| | Cycle Length | Length between burst sequences | | | | |
| PCLend | End Pacing | Ending cycle length between | calculated | | | msec |
| | Cycle Length | pacing pulses for a particular | = PCLstart - (PCLfactor * | | | |
| | | burst sequence | CLstddev) - [(N-1) * | | | |
| | | This formula will cause pacing | dPCLend * CLstddev] | | | |
| | | to end at a higher heart rate than | where N = burst sequence | | | |
| | | the starting rate. The ending rate | number | | | |
| | | will be increased further during | conditions: | | | |
| | | each subsequent burst sequence. | (PCLstart - PCLend) ≧ | | | |
| | | The standard deviation of the | dPCLmin | | | |
| | | cycle length is used as a | [PCLend(N-1) - PCLend(N)] | | | |
| | | modifier so that more irregular | ≧ dpCLendmin | | | |
| | | rhythm results in a higher | | | | |
| | | ending pacing rate | | | | |
| dPCLmin | Minimum | Minimum decrement in Cycle | 0 | 100 | 10 | msec |
| | Cycle Length | Length ramp between PCLstart | | | | |
| | decrement | and PCLend | | | | |
| dPCLendmin | Minimum | Guaranteed minimum decrement | 10 | 40 | 10 | msec |
| | Decrement of | in End Pacing Cycle Length | | | | |
| | End Pacing | between burst sequences | | | | |
| | Cycle Length | | | | | |
| PRDnom | Nominal | Nominal value of the duration | 1 | 500 | 50 | msec |
| | Pacing Ramp | for which the Pacing Cycle | | | | |
| | Duration | Length will be decreased | | | | |
| PRD | Pacing Ramp | Number of seconds during | calculated | | | sec |
| | Duration | which the Pacing Cycle Length | = PRDnom * CLstddev / | | | |
| | | is linearly decreased (pacing rate | 1000 | | | |
| | | is increased) | conditions: | | | |
| | | The nominal value is modified | PRDmin ≦ PRD ≦ PRDmax | | | |
| | | by the standard deviation of the | | | | |
| | | cycle length so that more | | | | |
| | | irregular rhythm results in | | | | |
| | | longer pacing duration | | | | |
| PRDmin | Minimum | Guaranteed minimum duration | 0.5 | 10 | 1 | sec |
| | Pacing Ramp | of the frequency ramp | | | | |
| | Duration | | | | | |
| PRDmax | Maximum | Guaranteed maximum duration | 1 | 100 | 10 | sec |
| | Pacing Ramp | of the frequency ramp | | | | |
| | Duration | | | | | |
| PBT | Pacing Burst | Number of seconds to continue | 0 | 10 | 1 | sec |
| | “tail” | pacing at the End Pacing Cycle | | | | |
| | | Length after the ramp is fully | | | | |
| | | completed | | | | |
| ST | Sensing Time | Number of seconds to sample | 1 | 10 | 5 | sec |
| | | atrial arrhythmia | | | | |
| TNPS | Total Number | Total number of burst sequences | 1 | 10 | 5 | N/A |
| | of Pacing | in a particular test | | | | |
| | Sequences | | | | | |
| dTseq | Time between | Time between burst sequences | 1 | 60 | 5 | sec |
| | sequences | (must be ≧ ST) | | | | |
| OPW | Output Pulse | Output pulse width of pacing | 1 | 30 | 15 | msec |
| | Width | signal | | | | |
| OCL | Output | Output current level of pacing | 0.5 | 30 | 15 | mA |
| | Current Level | signal | | | | |

### Example 4

Analysis of the Effects of Transesophageal Pacing

A two-sided Fisher's exact test is used to compare the safety and efficacy of the two treatment arms. Comparisons of successful termination of atrial fibrillation will be made after the first five minutes (i.e., pacing to placebo), prior to the transthoracic shock stage (i.e., pacing-ibutilide-pacing to placebo-ibutilide) and after completion of the complete protocol (i.e., after cardioversion or transthoracic shock, if necessary). Assuming an ibutilide efficacy of 15% after 10 minutes (based on data collected at the Mayo Cardioversion Center), 60 individuals are required in each treatment arm to detect an increase in efficacy in the pacing arm of 25%. This is based on a Fisher's exact test with a 0.05 two-sided significance level and 80% power.

Logistic regression is used to determine if left atrial electrogram characteristics (cycle length, cycle length variability, and amplitude) are associated with pacing efficacy. Furthermore, multiple logistic regression is used to determine if the left atrial electrogram variables (cycle length, cycle length variability, and amplitude) are independently associated with pacing efficacy while adjusting for other variables such as age, gender, and degree of heart disease, if necessary. The outcome variable for the logistic regression models is the pacing efficacy (n=60). With a sample size of 60, the logistic regression test of the standardized β=0 (significance level=0.50, two-sided) has approximately 70% power to detect a standardized β of 0.9 (an odds ratio of 2.5). This statistical analysis assumes that a left atrial electrogram variable is the only covariate and that the proportion of successes is 0.40 at its mean value.

Simple linear regression is used to determine if left atrial electrogram characteristics (cycle length, cycle length variability, and amplitude) are associated with duration of the atrial fibrillation, left atrium size, and left atrial appendage average emptying velocity. Furthermore, multiple linear regression is used to determine if duration of the atrial fibrillation, left atrium size, and left atrial appendage average emptying velocity are independently associated with each of the left atrial electrogram characteristics while adjusting for other variables such as age, gender, and degree of heart disease, if necessary. The outcome variables for the linear regression models are the left atrial electrogram characteristics (n=120); each of the three left atrial electrogram characteristics are treated as an outcome variable and modeled separately. A 0.05 two-sided Fisher's z test of the null hypothesis that the Pearson correlation coefficient ρ=0 will have approximately 80% power to detect a ρ of 0.25 between a particular atrial electrogram variable and one of the clinical, structural, or hemodynamic variables of interest.

### Example 5

Cardioversion Using a Transesophageal Probe

The addition of electrodes also permits transesphageal cardioversion. Cardioversion from within the esophagus using a large surface area electrode may be particularly promising. The combination of a large surface area electrode with the close positioning of the probe relative to the atria leads to high efficacy treatment using low energy administration. Methods of cardioversion as described herein permit shock delivery without general anesthesia.

### Example 6

Cardioversion Waveform

Since atrial fibrillation is not immediately life threatening, a cardioversion waveform is designed to minimize pain, rather than guarantee success with single shock. The following are main characteristics of a waveform optimized for transesophageal therapy. Any or all of the following elements can be incorporated.

1) Ascending ramp (monophasic, biphasic, or multiphasic):

a) traditional descending ramps have a low voltage “tail” which can lead to “refibrillation” and consequent unsuccessful shocks;

b) to avoid refibrillation, descending ramp shock waveforms have been ultra short, or more commonly, truncated, wasting energy on the capacitor;

c) ascending ramps typically don't lead to refibrillation, which increases their effectiveness;

d) ascending ramps permit delivery of longer duration, lower peak voltage shocks. Shock pain has been correlated to the peak voltage delivered and shock effectiveness to the total energy delivered; an alternative application is long duration truncated descending pulses;

e) currently available shock waveforms typically have <10-20 msec duration. Long shocks (>20 msec <50 msec, 100 msec, 250 msec, 1000 msec or longer) may permit termination of atrial fibrillation without significant pain. Shock effectiveness is related to the total energy delivered (increased with long pulse width/duration); pain is related to the peak voltage (can be lowered while maintaining energy by use of long pulse duration).

f) the electronics required for ascending ramp waveform are traditionally “bulkier” than that for a descending ramp (capacitor discharge). The size of electrodes are not an issue for transesophageal therapy, because the electronics are not implanted.

2) Smoothed/curved shape of the waveform:

a) avoidance of “sharp” voltage peaks/edges can further reduce pain perception with shock delivery.

3) Tilt of the waveform:

a) the waveform design described herein does not restrict tilt, although an ascending ramp is best described as having “reverse” tilt, or an ascending slope. The recommended starting slope is 1 per phase. Experimental data, however, will be required for tilt (slope) optimization.

4) Voltage reversal (between phases):

a) the waveform design described herein restricts voltage reversal. The instantaneous voltage reversal, however, is defined by the maximum voltage of the first phase.

5) Phase duration (biphasic waveform):

a) any combination of phase durations are employed with the cardioversion waveform described herein. The recommended starting phase duration (phase 1:phase 2) is 60:40.

6) Polarity:

a) either polarity is compatible with the design described herein.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An esophageal probe for transesophageal cardiac stimulation, comprising:
(a) an elongated flexible member having a distal portion and a proximal portion, wherein said distal end is closed for inserting into the esophagus; and
(b) a plurality of conductive electrodes, wherein said plurality of electrodes are circumferentially disposed at least partially around the distal portion of the elongated member.

2. The probe of claim 1, further comprising at least one conductor extending from the proximal portion to the plurality of electrodes.

3. The probe of claim 1, wherein said plurality of electrodes are connected to one another.

4. The probe of claim 1, wherein said plurality of electrodes are distinct from one another.

5. The probe of claim 1, further comprising a circuit.

6. The probe of claim 1, further comprising a pulse generator.

7. The probe of claim 1, further comprising a selector means.

8. The probe of claim 7, wherein particular electrodes of the plurality of electrodes can be selected using said selector means.

9. The probe of claim 6, further comprising a control unit.

10. The probe of claim 9, wherein said control unit selectively varies at least one characteristic of a pulse from said pulse generator, wherein said characteristic is selected from the group consisting of the waveform, the duration of the pulse, the interval between pulses, and the sequence of pulses to various electrodes.

11. The probe of claim 1, wherein one or more of said plurality of electrodes function as pacing electrodes, defibrillation electrodes and/or sensing electrodes.

12. The probe of claim 1, wherein each of said plurality of electrodes are spaced longitudinally apart at a distance of from about 5 mm to about 5 cm.

13. The probe of claim 1, further comprising a temperature sensor at said distal end and at least one conductor extending from said open proximal end through said elongated member to said temperature sensor.

14. The probe of claim 11, wherein said pacing electrodes conduct electrical current at about 1 milliamp (mA) to about 20 mA.

15. The probe of claim 11, wherein said defibrillation electrodes emit energy of from about 1 Joule (J) to about 100 J.

16. An improved transesophageal echocardiogram probe including: a housing having a cylindrical cavity formed therein; an elongated, multi-element ultrasonic array, said array including a number of elongated piezoelectric elements having emitting surfaces arranged in a plane, said array having a scan axis which is perpendicular to the long axis of said elements, said array being mounted on a pulley within said cavity; means for rotating said pulley within said housing, whereby said array will be rotated relative to said housing and in the plane of said elements around the axis of rotation of said pulley; means adapted for connecting said means for rotating to an operator control remote from said housing; and means for electrically connecting said array to an external ultrasound unit, wherein the improvement comprises:
a plurality of conductive electrodes, wherein said plurality of electrodes are circumferentially disposed at least partially around a distal portion of the housing.

17. The improved transesophageal echocardiogram probe of claim 16, further comprising at least one conductor extending from a proximal portion of the housing to the plurality of electrodes.

18. The improved transesophageal echocardiogram probe of claim 16, wherein said plurality of electrodes are connected to one another.

19. The improved transesophageal echocardiogram probe of claim 16, wherein said plurality of electrodes are distinct from one another.

20. The improved transesophageal echocardiogram probe of claim 16, wherein said plurality of electrodes are disposed on a disposable membrane.

21. A pacing or defibrillating member usable with a transesophageal probe, said member comprising:
a flexible, planar sheet member comprising a plurality of spaced apart electrical conductors;
a layer of adhesive carried on said sheet member; and
a removable protective cover overlaying said adhesive layer, wherein said flexible, planar sheet member is configured for overlaying onto said transesophageal probe.

22. The pacing or defibrillating member of claim 21, wherein said flexible planar sheet member further comprises a plurality of spaced apart planar electrodes, wherein said plurality of spaced apart planar electrodes corresponds positionally to said plurality of spaced apart electrical conductors.

23. The pacing or defibrillating member of claim 21, further comprising a plurality of planar electrodes.

24. The pacing or defibrillating member of claim 23, wherein said electrodes comprise low impedance defibrillating electrodes.

25. The pacing or defibrillating member of claim 23, wherein said electrodes are pacing electrodes.

26. The pacing or defibrillating member of claim 21, wherein said member is disposable.

27. A method for treating atrial fibrillation, comprising the steps of:
a) inserting a flexible probe into the esophagus of an individual at a position within the esophagus wherein the probe is adjacent to the atrium of the individual's heart, wherein the probe comprises a plurality of electrodes connected to a pacing generator via at least one conductor; and
b) generating pacing pulses in said pacing generator,
c) transmitting said pacing pulses from said generator to one or more of said electrodes via said at least one conductor; and
d) transmitting said pacing pulses from said electrode(s) through the wall of said esophagus to said individual's atrium, thereby treating said atrial fibrillation in said individual.

28. The method of claim 27, wherein said pacing pulses are administered in a range of from about 75 mA to about 150 mA.

29. The method of claim 27, wherein said pacing pulses are administered at a rate of from about 70 to about 100 pulses per minute.

30. The method of claim 27, wherein said pacing pulses are administered at a frequency of 50 Hz.

31. A method of terminating atrial fibrillation in an individual's heart, comprising the steps of:
a) positioning a plurality of conductive electrodes in the individual's esophagus adjacent to a posterior surface of the heart; and
b) pulsing selected of the conductive electrodes with electrical signals of a predetermined frequency thereby terminating the atrial fibrillation

32. A method of cardioversion, comprising the steps of:
a) inserting a TE probe into the esophagus of an individual in need of cardioversion; and
b) emitting an electrical signal from said TE probe, wherein said electrical signal results in cardioversion.

33. The method of claim 32, wherein said signal is ascending in voltage.

34. The method of claim 32, wherein said signal is biphasic.

35. The method of claim 32, wherein said electrical signal is a rounded biphasic ascending ramp.

36. The method of claim 32, further comprising the step of:
obtaining a cardiac image on said individual prior to or concurrent with said emission of said electrical signals.

37. A system for performing electrophysiological testing on an individual, comprising:
a) the probe of claim 1;
b) a pulse generator and receiver means connected to at least two of said plurality of electrodes for delivering pulses to selected electrodes and for receiving electrical signals induced in selected electrodes;
c) control means connected to said pulse generator and receiver means for controlling generation of the pacing pulses;
d) monitoring means connected to said pulse generator and receiver means for displaying data representative of parameters of electrical signals induced in at least one of the plurality of electrodes.

38. The system of claim 37, further comprising means for displaying an electrocardiogram representing sensed electrical activity of the heart.

39. A computer readable storage medium having instructions stored thereon causing a programmable processor to:
(1) determine local bipolar cycle lengths, minimum cycle length, maximum cycle length and mean cycle length over 5 sec;
(2) administer electrical signal beginning at maximum cycle length and, over 2 sec, accelerate to the mean cycle length which is maintained for 2 sec;
(3) repeat step (1);
(4) if atrial fibrillation is present, continue to step (5); if atrial fibrilliation is absent, stop;
(5) administer an electrical signal beginning at maximum cycle length and, over 2 sec, accelerate to a cycle length ½ the distance from the mean to the minimum cycle length which is maintained for 2 sec;
(6) repeat step (1);
(7) if atrial fibrillation is present, continue to step (8); if atrial fibrillation is absent, stop;
(8) administer an electrical signal beginning at maximum cycle length and, over 2 sec, accelerate to the minimum cycle length which is maintained for 2 sec;
(9) repeat step (1); and
(10) if atrial fibrillation is present, repeat steps (8), (9) and (10); if atrial fibrillation is absent, stop.
